(19) United States
(12) Patent Application Publication
(10) Pub. No.: US 2007/0269840 A1
(43) Pub. Date: Nov. 22, 2007

(54) In-situ equilibrium dialysis

(75) Inventors: Charles R. Carpenter, Scarborough, ME (US); Giosi Farace, Georgetown, ME (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IDEXX Laboratories, Inc.

(21) Appl. No.: 11/437,203

(22) Filed: May 19, 2006

(51) Int. Cl. G01N 33/53

(52) U.S. Cl. 435/7.92

(57) ABSTRACT

A method for the determination of analytes in samples using a pre-precipitated antibody complex. The complex includes an anti-analyte antibody and a secondary binding molecule that results in the precipitation of the antibody. Labeled-analyte analog competes for binding sites on the complex with analyte in the sample. The presence or amount of the analyte in the sample can is determined by detecting the amount of the label associated with the complex. The pre-precipitated antibody complex may be preloaded with labeled-analyte analog.

### BACKGROUND OF THE INVENTION

1. Field of the Invention

The invention is related to a method and kit for detecting analytes in biological samples. More particularly, the invention relates to a detection method and kits that use a pre-precipitated antibody complex.

2. Description of Related Art

Various analytical procedures and devices are commonly employed in immunoassays to determine the presence and/or amount of substances of interest or clinical significance which may be present in biological or non-biological fluids. Such substances are commonly termed “analytes” and routinely include substances such as antigens, drugs, hormones and the like.

The ability to use materials which specifically bind to an analyte of interest has created a burgeoning diagnostic device market based on the use of immunoassays. Immunoassays incorporate specific binding members, typified by antibody and antigen immunoreactants, wherein one member of the specific binding pair is labeled with a signal-producing compound (e.g., an antigen labeled with a fluorescent compound, a chemiluminescent compound, or a radioactive isotope.). In one typical “competitive” immunoassay, the test sample suspected of containing analyte can be mixed with a labeled-analyte analog (i.e., a conjugate), and incubated with an anti-analyte antibody for a period of time sufficient for a specific binding immunoreaction to occur. The reaction mixture is subsequently analyzed to detect either the presence of amount of the label associated with an anti-analyte antibody (bound conjugate). As a result, the amount of label bound to the antibody can be correlated to the amount of analyte in the test sample.

One of the challenges of immunoassay techniques is the measurement of an analyte that exists in a sample in both free and bound forms where, in the bound form, the analyte is bound to a receptor or other substance that is present the sample. The substance present in the sample that binds the analyte complicates the detection of the analyte because the substance interferes with the binding of the analyte to the anti-analyte antibody that is used as the capture reagent in the immunoassay. Interfering substances include, for example, receptors or anti-analyte antibodies naturally present in the sample.

Equilibrium dialysis is one well regarded method of separating bound analytes from free. The method relies generally upon the principle that the free analyte can diffuse through a membrane while the bound analyte can not. The amount of free analyte can then be measured without the presence of the interfering substance. Equilibrium dialysis has been used to provide an accurate characterization of a candidate drug in serum binding assays or in detailed studies of antigen-antibody interactions. Since the results of the assay following equilibrium dialysis are obtained under equilibrium conditions, the true nature of the interaction can be studied. In some instances, equilibrium dialysis also offers the ability to study low affinity interactions that are undetectable using other methods.

Using equilibrium dialysis, however, does not provide an efficient format desired in the clinical laboratory setting. It is generally considered to be a cumbersome procedure, difficult to do, and with few exceptions that include specially designed apparatus, it generally entirely outside the purview of routine clinical chemistry.

For instance, equilibrium dialysis is a technically demanding and time-consuming assay to perform. Even in the hands of a skilled operator the technique results in high variation when samples are subjected to repeated analysis. Accordingly, the inventors have recognized a need in the prior art to develop a simple and accurate method for determining, qualitatively and quantitatively, the amount of analyte in a sample when the analyte is bound to its receptor or other substance in the sample.

### SUMMARY OF THE INVENTION

In one aspect, the invention is directed to a method for determining a free analyte in a biological sample having the analyte bound in equilibrium with a binding partner for the analyte in the sample. The method includes contacting the sample containing the analyte in both free and bound forms with a complex including an antibody in precipitated form wherein the antibody substantially binds the analyte. The sample is incubated with the complex and then the complex is separated from the sample. The complex is then contacted with a solution containing a labeled-analyte analog. After the complex is separated from the solution, the presence or amount of the label associated with the complex is detected, which allows for the determination of the presence or amount of the free analyte in the sample.

In a further aspect, the invention includes a method for determining an analyte in a biological sample, where the method includes contacting the sample with a complex of a (i) a first antibody specific for the analyte in precipitated form and (ii) a labeled-analyte analog, wherein the labeled-analyte analog is specifically bound to the first antibody in precipitated form. The complex and the sample are incubated, and the complex is then separated from the sample. The presence or amount of the label from the labeled-analyte analog associated with the complex is detected, which allows for the determination of the presence or amount of the analyte in the sample.

In various aspects of the invention, the complex includes a macromolecule that specifically binds the first antibody, or the complex includes a second antibody that is specific for a first antibody. In another aspect, the sample has not been treated with an agent that releases the analyte from its natural binder partner in the sample. Also, one variation of the invention includes a method where the sample and the complex are incubated for a time sufficient to equilibrate the binding of the analyte to the complex and to its receptor in the sample. The label on the labeled-analyte analog may be sufficiently small to allow for the labeled-analyte analog to bind to binding sites throughout the complex.

Still further, the invention is directed to a preformed reagent, and a kit including the reagent, for the detection of an analyte comprising a complex of a precipitated antibody wherein the binding sites on the antibody are occupied by a labeled-analog of the analyte.

Yet another aspect of the invention is directed to a method for preparing a pre-precipitated antibody complex for use in an immunoassay for an analyte of interest in a sample. In one embodiment, the method includes incubating an antibody specific for the analyte with a labeled-analyte analog for a period of time sufficient to saturate the antibody with labeled-analyte analog, and precipitating the antibody by contacting the antibody with a macromolecule that binds to the antibody and causes the precipitation of the pre-precipitated antibody complex. Another embodiment of the method includes precipitating an antibody specific for the analyte by contacting the antibody with macromolecule that binds to the antibody and causes the precipitation of a complex of the antibody and the macromolecule, and incubating the complex with a labeled-analyte analog for a period of time sufficient to saturate the antibody with labeled-analyte analog, thereby forming the pre-precipitated antibody complex.

### BRIEF DESCRIPTION OF THE FIGURES

FIG. 1 is a standard curve created with known concentrations of free T4.

FIG. 2 is a graph showing the results of assays of samples from apparently healthy dogs for free T4 using radio-labeled T4 and a pre-precipitated antibody complex of the invention.

FIG. 3 is a graph showing the amount of free T4 in samples from apparently healthy dogs using an iodinated pre-precipitated antibody complex of the present invention.

FIG. 4 is a standard curve created with known concentrations of free T4 and a pre-loaded (iodinated), pre-precipitated antibody complex of the invention.

FIG. 5 is a graph showing the amount of free T4 in samples from apparently healthy dogs using an iodinated pre-precipitated antibody complex of the present invention.

### DETAILED DESCRIPTION

In general, the invention relates to a method for detecting an analyte in a biological sample. The invention is particularly useful for, but not limited to, detecting analytes that are reversibly bound to their receptors in the biological sample. For example, many biological samples include both the analyte and an antibody or other binding partner for the analyte. Free analyte is present in the sample in an amount depending upon the analyte's affinity for its receptor. When the sample is incubated with a pre-precipitated complex including an antibody specific for the analyte, the free analyte will bind with the complex. The nature of the complex prevents, to a large extent, the possibility of any individual analyte molecule from binding both the complex and its natural binding partner in the sample. The binding of the free analyte to the complex can be detected and, therefore, the presence or amount of the analyte in the sample can be determined.

Before describing the present invention in detail, a number of terms will be defined. As used herein, the singular forms “a,” “an”, and “the” include plural referents unless the context clearly dictates otherwise.

By “analyte” is meant a molecule or substance to be detected. For example, an analyte, as used herein, may be a ligand, which is mono- or polyepitopic, antigenic or haptenic; it may be a single compound or plurality of compounds that share at least one common epitopic site. The invention is appropriate for most small molecules with a molecular weight of a few thousand such as drugs and small molecule hormones that are capable of diffusing into the pre-precipitated complex.

A “sample” refers to an aliquot of any matter containing, or suspected of containing, an analyte of interest. For example, samples include biological samples, such as samples from taken from animals (e.g., saliva, whole blood, serum, and plasma, urine, tears and the like), cell cultures, plants, etc.; environmental samples (e.g., water); and industrial samples. Samples may be required to be prepared or pretreated prior to use in the methods of the invention. For example, where the sample is initially complex, solid, or viscous, it can be extracted, dissolved, filtered, centrifuged, stabilized, or diluted in order to obtain a sample having the appropriate characteristics for use with the invention. For the purposes herein, “sample” refers to the either the raw sample or a sample that has been prepared or pre-treated.

A “natural binding partner for the analyte” refers to a binding partner for the analyte that may be present in the sample. The natural binding partner may be an antibody or other receptor that may be present in raw samples or remaining after a sample has been pretreated or otherwise prepared. Most often, the natural binding partner will be naturally present in samples taken directly from the test subject. For the purposes herein, however, it should be understood that the natural binding partner may be artificial or synthetic, for example recombinant antibodies or other binding partners for the analyte that may be combined with the analyte, for whatever reason, before the sample is tested. Substances that non-specifically bind the analyte, from whatever source, are also included in this definition.

“Binding specificity” or “specific binding” refers to the substantial recognition of a first molecule for a second molecule, for example a polypeptide and a polyclonal or monoclonal antibody, or an antibody fragment (e.g. a Fv, single chain Fv, Fab′, or F(ab′)2 fragment) specific for the polypeptide.

“Non-specific binding” refers to non-covalent binding between molecules that is relatively independent of specific surface structures. Non-specific binding may result from several factors including electrostatic and hydrophobic interactions between molecules.

“Member of a specific binding pair” or “specific binding partner” refers one of two different molecules, having an area on the surface or in a cavity which specifically binds to and is thereby defined as complementary with a particular spatial and polar organization of the other molecule. The members of the specific binding pair are often referred to as ligand and receptor (antiligand). These will usually be members of an immunological pair such as antigen-antibody, although other specific binding pairs such as biotin-avidin, hormones-hormone receptors, IgG-protein A, and the like are not immunological pairs but are included in the invention and the definition of specific binding pair member.

“Analyte-specific binding partner” refers to a specific binding partner that is specific for the analyte.

“Substantial binding” or “substantially bind” refer to an amount of specific binding or recognizing between molecules in an assay mixture under particular assay conditions. In its broadest aspect, substantial binding relates to the difference between a first molecule's incapability of binding or recognizing a second molecule, and the first molecule's capability of binding or recognizing a third molecule, such that the difference is sufficient to allow a meaningful assay to be conducted distinguishing specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, preferably less than 10%, more preferably less than 5% of the reactivity exhibited toward a third molecule under a particular set of assay conditions, which includes the relative concentration and incubation of the molecules. Specific binding can be tested using a number of widely known methods, e.g., an immunohistochemical assay, an enzyme-linked immunosorbent assay (ELISA), a radioimmunoassay (RIA), or a western blot assay.

“Ligand” refers any organic compound for which a receptor naturally exists or can be prepared.

“Hapten” refers to a small molecule that reacts specifically with an antibody but cannot induce the formation of antibodies unless it is bound to a carrier protein or other large antigenic molecule.

“Analyte analog” or an “analog of the analyte” refers to a modified form of the analyte which can compete with the analyte for a receptor, the modification providing a means to join the analyte to another molecule. The analyte analog will usually differ from the analyte by more than replacement of a hydrogen with a bond that links the analyte analog to a hub or label, but need not. The analyte analog can bind to the receptor in a manner similar to the analyte.

“Receptor” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, protein A, complement component C1q, and the like.

“Amount” means the concentration or quantity or a substance either relatively or absolutely.

“Antigen” means any substance that binds specifically to an antibody.

“Conjugate” means a compound that comprises two substances, wherein one of the substances is coupled to the other. Coupling can be covalent or non-covalent. An analyte analog is often made into a conjugate with the label and often referred to herein as the labeled conjugate.

“Antibody” refers to an immunoglobulin that specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular molecule is maintained.

A “label” is a molecule that is bound (via covalent or non-covalent means, alone or encapsulated) to another molecule and that is chosen for specific characteristics that allow detection of the labeled molecule. For example, suitable labels should be capable of conjugation with antigens and haptens in order to be used in the labeled conjugate. Selection of the label is based on size, synthetic convenience, emission maximum, quantum efficiency, stability under the assay conditions. The type of signal generated by the label (e.g., radiation, fluorescence, chemiluminescence), and the apparatus necessary for detecting the signal should also be considered when selecting the appropriate label.

Generally, the label is conjugated to a specific binding partner. The labels can be conjugated to haptens, antigens, ligands other binding partners to form a labeled conjugate, such a labeled-analyte analog, using any convenient method (see e.g. Harlow, E. & Lane, D. (1988)Antibodies: A Laboratory Manual, Cold Spring Harbor Laboratory, Cold Spring Harbor: N.Y.; Harlow, E. & Lane, D. (1999) Using Antibodies: A Laboratory Manual, Cold, Spring Harbor Laboratory Press, Cold Spring Harbor: N.Y.; Sambrook, J. et al. (1989) Molecular Cloning: A Laboratory Manual, Cold Spring Harbor Laboratory Press, Cold Spring Harbor: N.Y.; and the like). The attachment of the labels to binding partners may be accomplished directly, through a linker, or through a pair of specific binding partners as is well known in the art. While all of these methods are contemplated as part of the invention, whatever method that is used to couple the analyte and label should result in a relatively small size of the conjugate so that the conjugate can access the interior binding sites in the complex.

“Immunoassay” refers to a technique that makes use of the specific binding between an antigen and its homologous antibody, either polyclonal or monoclonal, in order to analyze for an analyte in a sample.

A “reagent” refers to a substance that participates in a chemical reaction or physical interaction. A reagent can comprise an active component, that is, a component that directly participates in a chemical reaction (e.g. covalent binding) or physical interaction (e.g. non-covalent binding), such as a labeled conjugate, and other materials or compounds directly or indirectly involved in the chemical reaction or physical interaction. It can include a component inert to the chemical reaction or physical interaction, such as catalysts, stabilizers, buffers, and the like.

In one aspect, the invention is directed to a method of detecting an analyte in a sample using a complex of an antibody in pre-precipitated form. The complex of the pre-precipitated antibody provides multiple binding sites that are specific for the analyte where those binding sites are located on the “exterior” and the “interior” of the precipitated complex. Binding sites on the interior of the complex are accessible to the free analyte. Steric hindrance, however, prevents the binding of the analyte to the interior of the complex when the analyte is bound to its natural binding partner in the sample. Without wishing to be bound by any theory, it is understood that that the size of the natural binding partner prevents the analyte, when bound to its natural binding partner, from accessing binding sites on the interior of the complex. Accordingly, free analyte will bind to throughout the complex but analyte bound to its natural binding partner will not.

The steric hindrance between the interior of complex and the analyte also results in the equilibrium of the binding between the complex and the analyte to be shifted towards the analytes binding to the complex. It is understood that analyte that becomes bound to the interior of the complex is more likely to stay bound than the analyte bound to the exterior of the complex. Therefore, binding sites on the interior of the complex will have an artificially higher affinity for the analyte than the binding sites on the exterior of the complex. The affinity of the binding sites on the interior of the complex and the size exclusion properties associated with the interior binding sites create a condition that allows for the accurate detection of analytes bound in equilibrium to their natural binding partners in a sample.

The size exclusion properties of the antibody complex provide a means for preventing large molecules from accessing the interior space of the complex. While both the interior and exterior binding sites throughout the complex will recognize and specifically bind the analyte of interest, the random conformation of the complex creates interstitial spaces in the interior of the complex that are associated with the interior binding sites. While the size of the interstitial spaces are random, it is expected that the large majority of the interior space will be inaccessible to analytes that are bound to their natural binding partners in the sample, larger analytes, such as proteins and analogs of the analyte bound to large labels. On the other hand, exterior binding sites are available on the outside surfaces of the complex, although the surface is expected to be for the most part porous to those molecules of sufficiently small size. Binding sites on the exterior of the complex are accessible to analytes in any form.

In one aspect, the invention includes incubating the sample and the complex and then separating the complex from the sample. The incubation allows for free analyte in the sample to bind the complex. The binding of free analyte results in analyte in the sample that is bound to its natural binding partner to dissociate from its natural binding partner, creating additional free analyte that may become bound to the interior the complex. The amount of binding of the analyte to the complex and to its natural binding partner in the sample will depend on the affinity of the analyte for the complex and the natural binding partner.

The time for incubation of the sample and the complex generally range from about 15 minutes to overnight, depending upon the analyte and the sample. Using samples containing T4 bound to natural binders, such as albumin and TBG, it was found that about 50% of the available binding sites on the complex were occupied within 30 minutes at room temperature.

After incubation, the sample and the complex are separated. The complex is then contacted with the labeled-analyte analog. Binding sites on the complex left unoccupied by the analyte from the sample will bind to the labeled-analyte analog.

The complex is then separated from the labeled-analyte analog and the amount of label associated with the complex can be measured, which allows for the determination of the presence or amount of the analyte in the sample. When the complex is saturated with analyte, no labeled-analyte analog can be bound. Therefore, the absence of the label from the complex is an indication that the analyte is present in the sample. Similarly, the amount of analyte in a sample can be determined by measuring the amount of signal generated by the label on the complex. When the analyte is present in a relatively small amount in the sample, the signal from the complex will be relatively large. When the analyte is present in a large amount, but not enough to saturate the complex, the complex will provide a small signal.

In this aspect, the method of the invention is in the form of a competitive immunoassay, wherein a labeled conjugate competes with the analyte for binding sites on the pre-precipitated antibody complex. In the absence of analyte, the labeled conjugate will not have competition for binding sites on the complex. Thus, the amount of label bound to the complex is inversely proportional to the amount of analyte present.

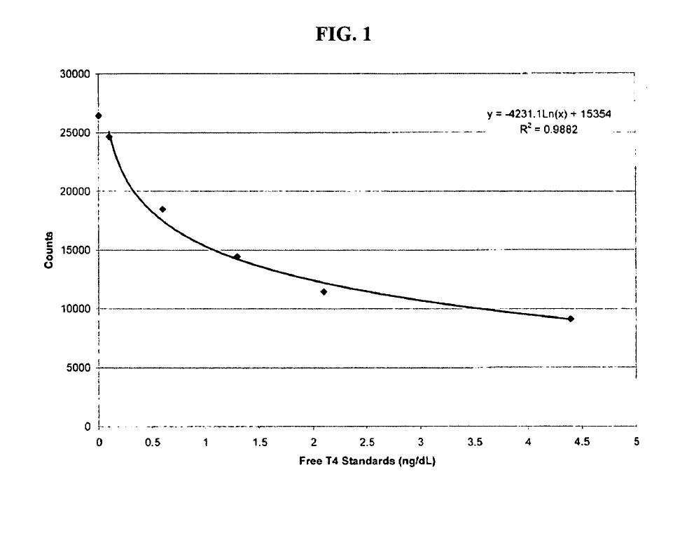

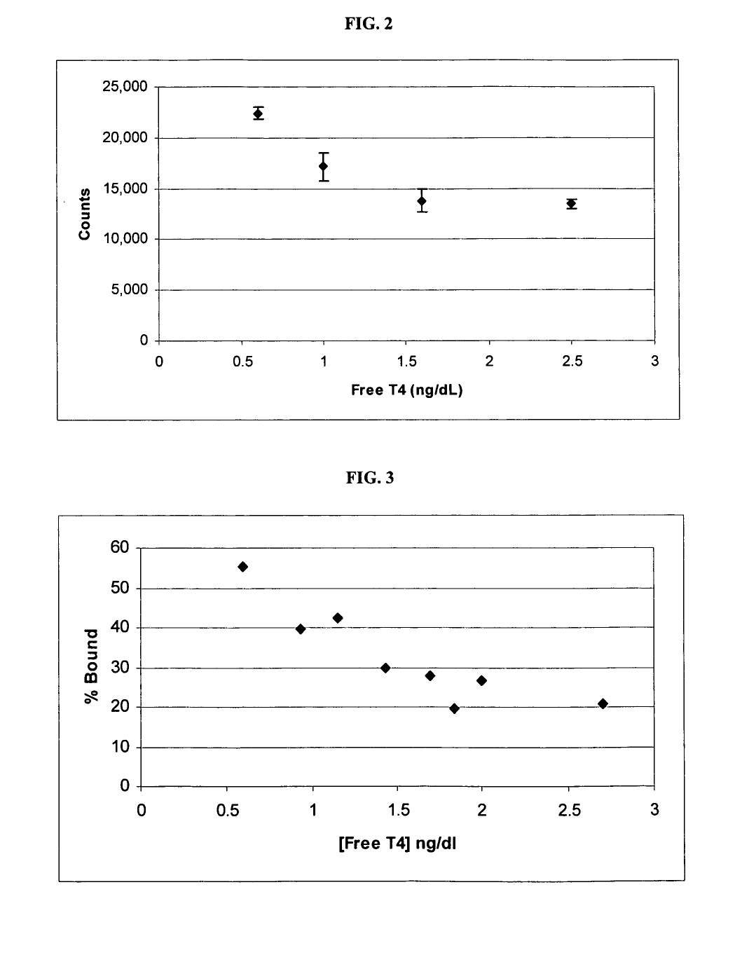

Using the appropriate calibrators and control, a standard curve can be established that relates the amount of signal from the complex to the amount of analyte in the sample. For example, FIG. 1 shows a standard curve produced from human free T4 standards. Various known concentrations of human T4 were incubated with a solution of pre-precipitated antibody complex having an anti-T4 antibody for 6 hours. The mixture was then centrifuged and the pellet was homogenized and incubated with ¹²⁵I-T4 labeled for 1 hour. The mixture was then centrifuged and the supernatant discarded to remove unbound labeled T4. The amount of the label associated with the complex was detected using a gamma counter.

The pre-precipitated antibody complex of the invention includes a primary antibody and a secondary binding macromolecule that may or may not be an antibody. Non-antibody binding macromolecules may include molecules which specifically bind the Fc region of immunoglobulins and capable of forming a three-dimensional network and an insoluble precipitate with the primary antibody. Macromolecules that bind to and precipitate antibodies are well known. The resulting complex should not clump, and preferably remain suspended in solution.

In another aspect, the invention relates to a pre-precipitated antibody complex that includes primary precipitated antibody that has been pre-blocked with labeled-analyte analog. In one aspect, pre-blocking is accomplished by incubating a pre-precipitated antibody complex with a labeled-analyte analog for a period of time that allows for the analyte binding sites on the precipitated complex to become saturated with labeled-analyte analog. Generally this can be accomplished with 30 minutes to overnight. Following the incubation, the complex can be pelleted, washed and homogenized for use in the method of detecting an analyte.

In another aspect, the pre-blocked, pre-precipitated antibody complex is formed by incubating the anti-analyte antibody with a labeled-analyte analog. Following incubation to saturate all of the antibody binding sites with labeled-analyte analog, the antibody is contacted with a secondary binding molecule that binds the antibody to form a precipitate.

Using the pre-blocked precipitated complex in the methods of the invention, sample is incubated with the complex from about 30 minutes to overnight and then separated from the complex. The incubation time allows analyte from the sample to exchange with labeled-analyte analog and thus decrease the amount of labeled-analyte analog in the complex. The change in labeled-analyte analog will therefore be proportional to the amount of analyte originally in the sample.

In the various aspects of the method of the invention, the complex is separated from the sample or labeled conjugate through centrifugation, decantation, filtration, and similar method, of the tube containing the complex. Centrifugation is generally performed from 10 to 30 minutes and at speeds from 5,000 to 15,000 rpm.

When unbound labeled conjugate is separated from the complex, the presence or amount of labeled conjugate remaining bound to the complex, or the unbound label, is detected in a detection step. The detection step can be conducted immediately after the separation step, or can be delayed for a period of time, if necessary. If the detection step is to be delayed, the complex and/or supernatant solution with the unbound labeled conjugate can be stored for a reasonable period of time under ambient or reduced temperature conditions.

Because the pre-precipitated complex can be removed from solution by centrifugation, filtration, decantation and similar methods, the primary antibody does not need to be bound covalently or noncovalently, physically or chemically to a solid phase which is insoluble in aqueous buffers. Thus, in this aspect, the components of the preformed complex are free from such materials as water insoluble organic polymeric substances including cellulose or other polysaccharides; vinyl addition polymers or condensation polymers such as aminoplasts or polyesters; water insoluble inorganic substances of polymeric nature such as glass or silicone resins; or solid supports such as polystyrene or polypropylene.

The primary antibody, and the secondary binding macromolecule when it is an antibody, may include the well known immunoglobulins, including IgG, IgM, IgA, IgD, and IgE, although IgG is most common. The antibodies may be prepared by any known method and the following examples of antibody preparation are not intended to limit the scope of the invention. For most analytes, the primary antibody can be purchased from commercial sources, with the exception that antibodies that do not immunoprecipitate should be avoided. Most polyclonal antibodies can be used in complexes using the appropriate antispecies antibody. For example, rabbit/antirabbit and sheep/antisheep antibodies form useful precipitates.

One method of obtaining the secondary antibody, involves raising the same in an appropriate animal, by injection into said animal of antibody-producing amounts of the Fc fragment of the primary antibody, or intact primary IgG, wherein said primary antibody is normally from a different species. Alternatively, the secondary antibody may be a monoclonal antibody obtained from a hybridoma or from the ascites fluid of an appropriate animal.

The preparation and isolation of Fc fragments from immunoglobulins, or of immunologically functional products of immunoglobulins which contain the Fc portion are known processes. For example, after sensitization and antibody formation in the second animal, the animal is bled and the secondary antibody, having substantial affinity for the Fc fragment is purified therefrom. Purification of the antibody needs to be carried out by affinity chromatography; a pre-purification, such as for example by ammonium sulfate precipitation may facilitate the procedure.

Affinity chromatography or immunoabsorbent chromatography of antibodies is a well known technique. An insoluble solid phase is prepared, and the Fc fragment or an immunologically functional product of immunoglobulin with immunologically functioning Fc is bound to the solid phase, preferably by covalent immobilization. The attachment of proteinaceous materials to resins is a well known technique in the art. The impure or partially purified antibody is brought into contact with the solid phase-bound Fc fragment, and binds thereto. When immunization has been carried out with Fc fragments the affinity column may contain intact IgG bound thereto. Antibodies and other materials which do not have specificity for the Fc fragment do not bind to the solid phase and remain in solution. The solid phase carrier or resin can be contacted with the solution containing the mixture of antibodies either in batch or in a column process. After separation of the original solution and washing of the carrier, the adsorbed secondary antibody is separated from the carrier by a solution containing a desorption agent such as an acid, a base or a high salt solution. After collecting fractions which contain the secondary antibody, the same can be freed of salts, acids or bases by dialysis, diafiltration or similar methodology.

Because human IgG present in a sample may, (when a secondary macromolecule has substantial cross reactivity with human IgG) may dissociate or tend to dissociate preformed complexes, it may be desired to remove from the affinity purified secondary binding macromolecule, any cross-reactivity with human IgG. In such instances, the affinity purified secondary macromolecule is further contacted (in batch or column) with solid phase-bound human IgG. The bound material is discarded, while the non-bound material is collected and used.

Alternatively, the secondary antibody can be raised in an animal by injecting into the animal whole primary antibody. This produces in the animal a family of secondary antibodies most of which have specificity for the Fc region of the primary antibody, and some of which have specificity for the binding regions (F(ab)) of the primary antibody. After prepurification by ammonium sulfate precipitation if necessary, this mixture of antibodies is purified by immunoaffinity chromatography as previously described, against a solid phase containing covalently bound Fc fragments or immunologically functional products of immunoglobulins containing functional Fc fragments.

It may be obtained by direct immunization schedules with the immunogen or from hybridomas or ascites fluid of appropriate animals. Immunization schedules are well known in the art. Immunogens for which a test is desired can be injected with adjuvant into antibody-producing animals such as horses, goats, rabbits, guinea pigs, an the like. Materials of low molecular weight (e.g., steroid hormones) are generally non-immunogenic, but become so when conjugated as “haptens” to larger molecules such as albumin. Factors which may enhance the response to a conjugate include a high density of hapten on the carrier, or the use of a carrier which is itself immunogenic.

In the vast majority of immunization schedules the antigen is injected as an emulsion in “complete Freund's adjuvant”. This is a mixture of mineral oil, detergent and killed mycobacteria. An immunization program, as is well known in the art, will yield a number of anti-sera from which one or more must be selected for use in an assay. The criteria for this selection are generally specificity, affinity and titer. The antibodies having the desired specificity and affinity can be prepurified by ammonium sulfate precipitation. In some cases affinity chromatography on a column or batch containing the immunogen or antigen covalently bound to a solid phase is used to further purify the antibody.

The complex of secondary binding macromolecule and primary antibody is formed before the preparation of the assay. A solution of secondary binding macromolecule is added to primary antibody in an appropriate physiological buffer (Tris, Hepes, Pipes, imidazole, glycerol phosphate, etc.). The rate of addition should be sufficiently slow to prevent flocculation of the complexes. For example, addition at a rate of about 1-2 minutes/mg of primary antibody is sufficient. After completing addition, the suspension is incubated at 4-30° C., preferably room temperature, for a period sufficient to cause substantially complete formation of the complex and precipitation thereof. A period of 1-4 hours is generally sufficient. The material is then incubated at 4° C. for about 24 hours. The ratio of secondary binding macromolecule to primary antibody is normally that which is sufficient to completely precipitate the primary antibody, such as 4.5-50:1, normally 10-15:1. Also, prior to the formation of the complex, the primary antibody may be pre-blocked with a labeled-analyte analog. Once formation and precipitation of the complex has occurred, the same remains suspended in the original buffer. The complex can be frozen or stored refrigerated until required.

Many different types of labels can be employed as long as they are sufficiently sized such that the labeled conjugate can access the interior binding sites of the complex. In general radiolabels are useful because of their small size and strong signal. Molecules useful as radio-labeled analyte analogs are available from a variety of commercial sources (e.g., ¹²⁵I T4 (˜5 million CPM) (Perkin Elmer)). In addition, preparation of radiolabeled molecules is within the skill in the art.

Selection of the fluorescent label is based on size, synthetic convenience, emission maximum, quantum efficiency, stability under the assay conditions, and the like, but the particular fluorescent label is not critical, so long as it is sufficiently sized and there is a minimum quantum yield to provide the desired sensitivity. A large number of commercially available fluorescent labels can be employed. Illustrative fluorescent labels include fluorescein-isothiocyanate (FITC), rhodamine, Texas Red, Cy-5®, and particularly, fluorescent labels that fluoresce above about 550 nm, more particularly, fluorescent labels that fluoresce above 600 nm, and efficiently absorb light having absorption above 500 nm; more particularly, 650 nm, such as Cy-5®. The fluorescent labels can be conjugated to form the labeled conjugate using any convenient method.

When fluorescently-labeled conjugates are used, they should be of sufficient size, for instance having a molecular weight below about 2K, to ensure that the labeled-analyte analog can access in the interior binding spaces of the complex. Examples of small fluorescent labels include Alexa fluors, which have molecular weights generally in the 700-800 range, and Cy dyes, which generally have molecular weights well under 1200. With fluorescent labels, detection is accomplished by first irradiating the complex, followed by measuring the resultant emitted fluorescent signal. Any convenient irradiation means can be employed for providing the appropriate wavelength. Exemplary irradiation means include lasers, light emitting diodes, tungsten lamps and the like. The wavelength of light used in the stimulation means will depend on the particular fluorescent label. Generally, the irradiation light wavelengths will range from 300 to 900 nm, usually from about 350 to 800 nm, and more usually from about 450 to 800 nm. The fluorescence from the fluorescently-labeled conjugates present in the complex can be measured. Measuring the emitted signal is accomplished by detecting the photons emitted from the complex. Means for measuring fluorescence are commercially available and any convenient fluorescence detector can be used. Various photodiodes, photomultipliers, and the like, can be employed, and in some instances a visual detection will suffice.

Chemiluminescent labels are also appropriate for the labeled-analyte analog, as long as the label is sufficiently sized. Useful chemiluminescent labels include, for example, acridinium esters, which generally have molecular weights of less then 1000. Other similarly sized chemiluminescent labels would be within the purview of the skill in the art. Detection of signals from chemiluminescent labels is well known, and it is not expected that the use of the appropriately sized labels in the present invention would vary significantly with the use of those labels in other immunoassays that employ them.

Generally only a small amount of the sample (less than about 1 ml) is employed in the various methods of the invention. It may be important that the mixing and incubation step and the subsequent incubation step in the method of this aspect of invention be carried out for the same defined period of time to minimize variations from test to test in a series. Thus, it may be preferred to automate the steps of the method to eliminate operator error to the greatest extent possible.

A variety of wash fluids can be used for the washing step. The pH of the wash fluid will be a pH in which the binding pair complexes are stable. Typically, the pH will range from 5 to 9, usually 6 to 8, and more usually about is 7. Depending on the nature of the label of the conjugate, wash solutions may which enhance the label can be employed. For example, the fluorescence of a particular fluorescent label can be enhanced in slightly alkaline or basic solution. In such a case, a buffer having a pH above 7, but usually less than 9, can be employed. Exemplary wash fluids comprise water, buffers, such as phosphate, phosphate buffered saline (PBS), saline solutions, carbonate buffers, and the like. The wash fluid can be introduced to the solid phase any convenient means. Usually the wash fluid will be introduced using the same means as the means used for introduction of the sample. To the extent that the substrate is a reaction well, the wash solution can be taken up a number of times, usually not more than about 6, more usually not more than about 2, or the wash solution can be forced through the well using a syringe, pump or other device.

In one aspect, the invention is directed to a method of measuring of thyroxine (3,5,3′,5′-tetraiodo thyroxine, T4), which has been a widely used test for thyroid function status. T4 circulates in serum primarily bound to the transport proteins thyroid binding globulins (TBG), albumin and prealbumin. A minute portion (˜0.03%) of total T4 is unbound (free T4). The unbound free T4 is the biologically active form which stimulates metabolism. Total T4 levels may rise or fall depending on the TBG level in euthyroid patients and can give the biochemical appearance of hypothyroidism or hyperthyroidism. Free T4 measurement is independent of carrier protein variation and more closely correlates the thyroid status. Free T4 clinically distinguishes euthyroid hyperthyroxinemias from hyperthyroidism and euthyroid hypothyroxinemias from hypothyroidism. Normally, circulating levels of free T4 range from 0.7 to 1.8 ng/mL. Hyperthyroidism is characterized by increased levels of free T4 and hypothyroidism by deceased levels. Along with TBG, other factors can influence the measurement of free T4, and include pregnancy, steroid hormones (estrogens and contraceptives) and drugs that displace the T4 from binding proteins such as phenytoin, phenylbutazone, salicylates and diphenylhydantoin. Accordingly, one aspect of the invention is directed to determining the thyroid function in a patient by detecting the level of the patients free T4.

Total T4 can be measured by releasing the T4 from its receptors with various reagents including ANS (8-anilino 1 naphthalene sulphonic acid), merthiolate, and the substances that displace T4 from its binding proteins. Without the release of T4 from its receptors in the sample, however, traditional immunoassays have not been sensitive enough to reliably detect free T4. Equilibrium dialysis methods have assisted in the detection of free T4 to some extent. In canines, however, T4 is bound with greater affinity for TBG than in humans. Therefore, smaller concentrations of free T4 are available in serum, and equilibrium dialysis methods have not been reliable to detect free T4. The present invention allows for the reliable detection of free T4 in canine samples.

In addition to T4, the invention is suitable for the detection of other analytes that are tightly bound to receptors or other binding partners in a sample These analyte include a number of therapeutic drugs, drugs of abuse, small molecule hormones such as cortisol and epinephrine, and countless other molecules of interest. The only limitation on the analyte is that it should be small enough so that a labeled-analyte analog can bind with generally the same affinity as the analyte to the interior binding sites of precipitated complex of an anti-analyte antibody.

Any or all of the above embodiments of the invention may be provided as a kit. In one particular example, such a kit would include a pre-precipitated antibody complex. The complex may be pre-loaded with labeled-analyte analog, or the labeled-analyte analog may be provided in the kit as a separate reagent. The kit may also include wash reagent and detector reagent, depending on the label employed. Positive and negative control reagents may also be included, if desired or appropriate. In addition, other additives may be included, such as stabilizers, buffers, and the like. The relative amounts of the various reagents may be varied widely, to provide for concentrations in solution of the reagents that substantially optimize the sensitivity of the assay. Particularly, the reagents may be provided as dry powders, usually lyophilized, which on dissolution will provide for a reagent solution having the appropriate concentrations for combining with the sample.

In one aspect, the invention includes an apparatus for determining the presence of at least one analyte in a sample, which apparatus comprises a reservoir such as a tube or well or microtiter plate, a means to control the flow of fluid contained within the reservoir, reader to measure the level of signal from the label(s) such as a gamma counter, fluorometer, CCD camera, spectrophotometer, or luminometer, and a means to analyze the resulting signal and report a qualitative, semi-quantitative or quantitative result.

The level of quantitation possible using the method described herein depends on the affinity of the binding partners as previously discussed, detector sensitivity, mathematics used to analyze the signal, and whether standards and/or controls are used and if so on what kinds of standards and/or controls.

Quantitative analysis is possible with the present invention. Again, referring for example to the use of labels, plots of normalized emission versus concentration of analyte. Normalized emission corresponds to the level of signal emitted by a label bound to the pre-precipitated antibody complex as a percentage of the level of signal emitted by the label bound to the complex with no analyte, i.e. a blank. Since the analyte concentration is inversely proportional to the level of signal for a comparative assay, the curves formed by the plurality of concentration points have a negative slope. If a sample is run on the apparatus, the resulting signal can be compared against the signal generated by a blank run in parallel with the sample. The resulting percentage can be plotted on the appropriate graph and a relative concentration of analyte in sample can be determined.

The apparatus can calculate a semi-quantitative result by using a pass/no-pass level of signal or a quantitative result by plotting a normalized level of signal versus concentration, and calculating a least-squares best fit of a line corresponding to the curves. Thus, a multilevel calibration curve can be used, wherein quantitative determination of the amount of analyte in a sample is possible when such concentration is interpolated within the linear range of the best fit polynomial. Even greater quantitative results are possible when standards are prepared to run in parallel with a sample immunoassay, wherein the same lot of label is used for both the sample conjugate and blank conjugate.

Positive and negative controls can be run with the assays, measurements, or tests disclosed herein. One convenient method is the use of a control for passing, failing, and blank concentrations of analyte. This control can be run before, during or immediately following a positive test result with the immunoassay.

Immunoassays that can be adapted for use with the present invention or variations thereof include RIA other immunoassays utilizing different labels, such as fluorescent and chemiluminescent compounds. Instrumentation can include custom or commercially available analytical instruments including, but not limited, to spectrophotometers, counters and other instruments necessary for a particular use. Conditions for use include, but are not limited to, clinical laboratories, use in the field, such as outdoors and/or on-site, and in other laboratory settings. Alternative embodiments of the present invention can thus be used to adapt to the above uses, conditions for use, and cooperation with other devices.

The following are provided for exemplification purposes only and are not intended to limit the scope of the invention described in broad terms above. All references cited in this disclosure are incorporated herein by reference.

### EXAMPLES

### Example 1

### Preparation of Pre-Precipitated Antibody Complex

Add dropwise 180 μg of rabbit anti-T4 (made in-house) to 1 mg of Fc purified goat anti-rabbit polyclonal (Jackson Immuno Research). Allow the mixture to stand at room temperature for 1-2 hours and then refrigerate overnight at 4° C. The next day, spin the complex for 5 minutes at 5,000 rpm and remove the supernatant. Resuspend the pellet in 5 ml of tris-buffered saline and store frozen or refrigerate follow the addition of 0.5% Kathon.

### Example 2

### Free T4 Assay Using Pre-Precipitated Antibody Complex

Mix 25 μl of canine serum with 25 μl of pre-precipitated complex of Example 1 and incubate at 37° C. for 3 hours. Dilute the mixture with 950 μl of 0.05M tris buffer containing 0.1% Sodium azide and 0.2 mM EDTA. Spin the solution at 10,000 rpm for 10 minutes and decant the supernatant. Add 950 μl of 5700 μCi/μg T4 I¹²⁵-T4 (Perkin Elmer) and incubate for 30 minutes at room temperature. Spin for 10 minutes at 10,000 rpm decant the supernatant and read the pellet in a gamma counter.

### Example 3

### Preparation Iodinated Pre-Precipitated Antibody Complex

Add 0.036 mg of rabbit anti-T4 to approximately 100 μl of 5700 μCi/μg T4 I¹²⁵-T4 (Perkin Elmer). QC to 1 ml with Tris buffer (4 mM Tris-HCl, 2 mM EDTA, 0.1% Sodium Azide, 0.1% Tween-20, pH9). Incubate at RT for 30 minutes.

Add 0.2 mg of purified goat anti-rabbit serum (Jackson Immuno Research) drop-wise to the antibody/T4 solution. Incubate at RT for 1-2 hours and then store overnight at 4° C.

Spin solution for 8 minutes at 13,000 rpm. Decant supernatant and wash the pellet with 1 ml of Tris buffered saline (TBS). Repeat a second time.

After the second wash, resuspend the pellet in 1 ml TBS containing 5 μl Kathon. Store at 4° C. until required.

### Example 4

### Free T4 Assay Using Iodinated Pre-Precipitated Complex

Prior to use, homogenize the pre-precipitated complex prepared as in Example 2 using a syringe and a narrow gauge needle. Expel and draw the solution through the needle vigorously 10-12 times and then vortex the solution for five seconds. The pre-precipitated complex is now ready to use

Add 10 μl of complex to each assay tube. Add 1 ml of Tris buffer and 100 μl of canine serum, vortex and incubate overnight at 37° C.

Read tubes in a gamma counter to get total counts. Spin tubes for 30 minutes at 13,000 rpm, save supernatant in a separate tube and read both supernatant and pellet in the gamma counter. Determine amount of binding of the labeled-T4 for each tube based on the ratio of the counts of the pellet and the total counts. FIG. 3 shows the results of this experiment with differing concentration of T4 in a sample. [Please send a new version without the n=2 on the axis]

### Example 5

### Pre Loading of Pre-Precipitated Antibody Complex with Labeled T4 Analog

To prepare a preloaded and pre-precipitated antibody complex, the precipitated complex should be prepared as in Example 1 and homogenized as described in Example 4.

To 1 ml of the non-labeled complex add approximately 15 μl of neat ¹²⁵I T4 (˜5 million CPM) (Perkin Elmer). Incubate at room temperature for 30 minutes. Following the incubation, centrifuge the mixture for 8 min. at 13,000 rpm. Decant and wash with 1 ml of TBS buffer twice.

After the second wash and decanting, resuspend the pellet with 1 ml of TBS buffer and 5 μl of kathon.

### Example 6

### Assay Protocol Using Pre-Load, Pre-Precipitated Anti-T4 Antibody Complex

To 1 ml of homogenized pre-loaded complex prepared as in Example 5, add 100 μl of canine serum diluted 1:9 using a 3% bovine IgG solution. Incubate the mixture overnight at room temperature.

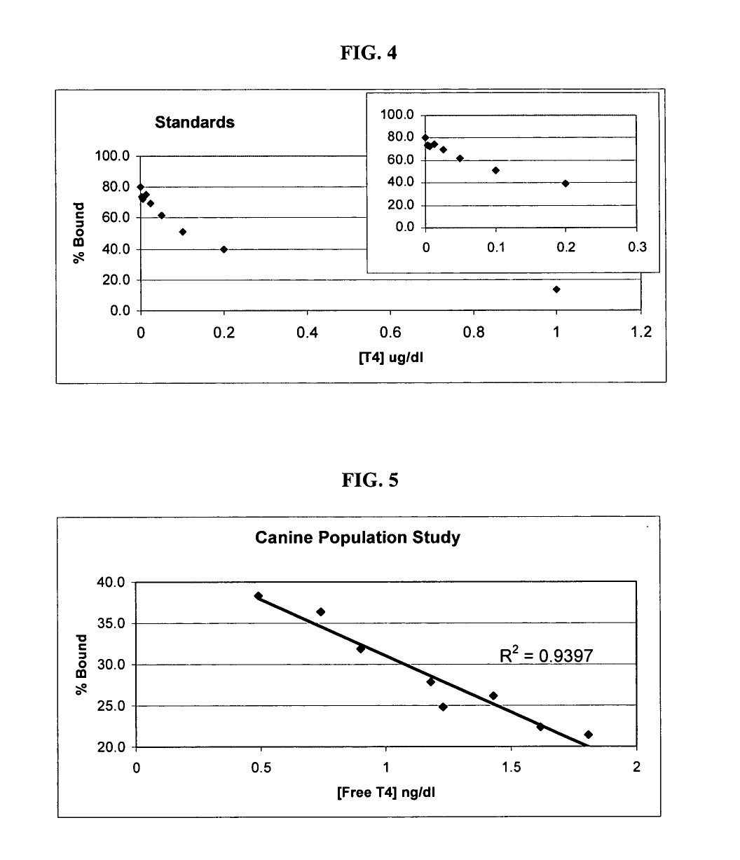

Following the incubation, read total counts of the tube. Spin for 30 minutes at 13,000 rpm and decant supernatant. Read the pellet and calculate percent of the total label bound. FIG. 4 shows a standard curve prepared by the above procedure using various known concentrations of labeled T4. FIG. 5 shows the results of the assay with the canine serum samples.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A method for determining a free analyte in a biological sample having the analyte bound in equilibrium with a binding partner for the analyte in the sample, the method comprising:
(a) contacting the sample containing the analyte in both free and bound forms with a complex comprising a first antibody in precipitated form wherein the first antibody substantially binds the analyte;
(b) incubating the sample and the complex;
(c) separating the complex from the sample;
(d) contacting the complex with a solution containing a labeled-analyte analog;
(e) separating the complex from the solution; and
(f) detecting the presence or amount of the label associated with the complex, thereby determining the presence or amount of the free analyte in the sample.

2. The method of claim 1 wherein the complex comprises the first antibody and a macromolecule that specifically binds the antibody.

3. The method of claim 2 wherein the macromolecule is a second antibody that is specific for the first antibody.

4. The method of claim 1 wherein the sample has not been treated with an agent that releases the analyte from its natural binder partner in the sample.

5. The method of claim 1 wherein the sample and the complex are incubated for a time sufficient to equilibrate the binding of the analyte to the complex and to its receptor in the sample.

6. The method of claim 1 wherein the label on the labeled-analyte analog is sufficiently small to allow for the labeled-analyte analog to bind to binding sites throughout the complex.

7. A method for determining the presence or amount of free analyte in a biological sample containing the analyte in both free and bound forms wherein the analyte is bound in equilibrium with a receptor for the analyte in the sample, the method comprising contacting the sample with a precipitated antibody specific for the analyte, allowing analyte to bind equilibrium to the antibody and its receptor, and determining the presence or amount of the analyte bound to the antibody.

8. The method of claim 7 wherein the determining of the presence or amount of the analyte bound to the antibody comprises separating the antibody from the sample, contacting the antibody with a labeled-analyte analog, and determining the extent of the binding between the antibody and the labeled-analyte analog.

9. A method for determining an analyte in a biological sample, the method comprising:
(a) contacting the sample with a complex comprising (i) a first antibody specific for the analyte in precipitated form and (ii) a labeled-analyte analog, wherein the labeled-analyte analog is specifically bound to the first antibody in precipitated form;
(b) incubating the sample and the complex;
(c) separating the complex from the sample; and
(d) detecting the presence or amount of the label from the labeled-analyte analog associated with the complex, thereby determining the presence or amount of the analyte in the sample.

10. The method of claim 9 wherein the complex comprises the first antibody and a macromolecule that specifically binds the antibody.

11. The method of claim 10 wherein the macromolecule is a second antibody that is specific for the first antibody.

12. The method of claim 9 wherein the sample contains free analyte and analyte bound to a receptor for the analyte in the sample, and wherein the sample and the complex are incubated for a time sufficient to equilibrate the binding of the analyte to the complex and to its receptor in the sample.

13. The method of claim 9 wherein the label is sufficiently small to allow for the labeled-analyte analog to bind to binding sites for the analyte throughout the complex.

14. The method of claim 9 wherein a signal from the complex is detected prior to contacting the complex with the sample.

15. A preformed reagent for the detection of an analyte comprising a complex comprising a precipitated antibody wherein the binding sites on the antibody are occupied by a labeled-analog of the analyte.

16. A kit for detecting an analyte bound in equilibrium with its natural binding partner in a sample, the kit comprising the preformed reagent of claim 15.

17. A method for preparing a pre-precipitated antibody complex for use in an immunoassay for an analyte of interest in a sample, the method comprising:
(a) incubating an antibody specific for the analyte with a labeled-analyte analog for a period of time sufficient to saturate the antibody with labeled-analyte analog, and
(b) precipitating the antibody by contacting the antibody with a macromolecule that binds to the antibody and causes the precipitation of the pre-precipitated antibody complex.

18. The method of claim 17 wherein the macromolecule is a second antibody that is specific for the first antibody.

19. The method of claim 17 wherein the labeled-analyte analog binds with substantially the same affinity as the analyte to binding sites throughout the complex.

20. A method for preparing a pre-precipitated antibody complex for use in an immunoassay for an analyte of interest in a sample, the method comprising:
(a) precipitating an antibody specific for the analyte by contacting the antibody with macromolecule that binds to the antibody and causes the precipitation of a complex of the antibody and the macromolecule,
(b) incubating the complex with a labeled-analyte analog for a period of time sufficient to saturate the antibody with labeled-analyte analog, thereby forming the pre-precipitated antibody complex.

21. The method of claim 20 wherein the macromolecule is a second antibody that is specific for the first antibody.

22. The method of claim 20 wherein the labeled-analyte analog binds with substantially the same affinity as the analyte to binding sites throughout the complex.
